(19) United States
(12) Patent Application Publication
(10) Pub. No.: US 2003/0170214 A1
(43) Pub. Date: Sep. 11, 2003

(54) Method for producing a bio-artificial transplant

(76) Inventor: Augustinus Bader, Immensen (DE)

Correspondence Address: CLARK & ELBING LLP, 101 FEDERAL STREET, BOSTON, MA, 02110, US

(21) Appl. No.: 10/296,671

(22) PCT Filed: May 28, 2001
(86) PCT No.: PCT/DE01/01986

(30) Foreign Application Priority Data
May 29, 2000 (DE) 10026482.4

(51) Int. Cl. A61K 48/00; C12N 5/08

(52) U.S. Cl. 424/93.21; 424/93.7; 435/368

(57) ABSTRACT

The invention relates to a method for producing a bioartificial transplant from biological tissue provided for transplantation, which has cells that are compatible with the recipient applied thereto. According to the invention, a controlled tissue generation is carried out in-vitro, during which selected cells that are capable of remodelling the carrier structures are added to native tissue that is maintained in a culture and the culture is continued until a new tissue which has been substantially transformed is obtained, said tissue containing the added recipient-compatible cells.

The invention relates to a process for the production of a bioartificial transplant from a biological tissue intended for transplantation and recipient-tolerable cells applied thereto.

The invention generally relates to a process for the controlled culturing of biological tissue.

In transplantation medicine, there is a great need for suitable transplants which cause adverse reactions in the transplant recipients to the lowest possible extent. Only in certain cases is it possible to remove the transplant from the body of the recipient himself and to transplant it. From the immunological point of view, these transplantations are the most acceptable, but in the case of certain vessels or organs and in the case of relatively large areas of skin to be replaced this possibility does not exist. For certain organs, today virtually only allogenic transplants of foreign donors or—frequently in the orthopedic field—synthetic implants of plastics, metals, ceramic etc. or various laminated materials are suitable. When using allogenic materials, such as, for example, donor organs, continuous immunosuppression which is stressing for the body of the recipient is necessary. Nevertheless, rejection reactions frequently occur as a serious complication. Plastic materials can also lead to rejection reactions and inflammatory processes, which destroy the operation result.

For various reasons, it is often attempted today to use xenogenic material (of animal origin). The better availability of this material is especially advantageous here compared with allogenic (donor) materials. Such a “biological material” is also more flexible than a plastic material and adapts better in some sites of the recipient's body. The xenogenic transplantation material, however, is therefore problematic, as it is strongly antigenic.

It has therefore been attempted for a relatively long time to make xenogenic transplantation materials—especially various tissue intended for transplantation—tolerable tolerable to the recipient. For this, as a rule it is attempted to destroy or to remove native cells within or embedded on the structure-imparting connective tissue matrix of the xenogenic transplant, and to wash out foreign proteins and other foreign substances. The structure-imparting matrix of interstitial connective tissue can be regarded as an immunologically largely neutral matrix.

Chemically treated transplants of animal origin are used, for example, for heart valve replacement in humans. The animal material is in this case in general treated with glutaraldehyde in order to stabilize the structural proteins and to prevent an antigenic reaction. The tissue treated with glutaraldehyde, however, undergoes continuous hardening and progressive calcification after transplantation. These transplants must therefore be replaced every few years.

As an alternative, it has also already been attempted to transplant an acellularized and thereby “neutralized” exposed collagen matrix, which, however, as shown, is likewise accompanied by problems. The acellularized collagen matrix is severely loosened by the acellularization process and mechanically unstable. A serious disadvantage of the destabilization is the danger of initial failure after implantation in the body due to rupture. This occurred in animal models in 20% of the cases (acellularized pig heart valves, recolonized with autologous cells in the low-pressure circulation, pulmonary position).

An exposed collagen matrix in the body is additionally easily attacked by collagenases, such that damage can occur before recolonization in the body could take place.

It has therefore likewise already been attempted to recolonize acellularized biological tissue intended for transplantation before the transplantation with autologous or allogenic cells. In DE 19828726 A1, for example, a process for the production of a bioartificial transplant is described, in which firstly native cells on the interstitial connective tissue of the transplant are destroyed and then removed. The matrix is then newly colonized with cells which are tolerable for the recipient, preferably autologous cells, so that a recipient-specific biotransplant is obtained.

It is already very advantageous here that antigenic components are largely removed or screened. The bioartificial transplant obtained in this manner, however, still does not have the required “natural” properties. The growth of the cells on the acellularized loosened matrix is made difficult. By means of an even small change in the matrix structure, a completely natural reconstruction of the cells is also not obtained. There are also still considerable problems in controlling the necessary growth of various differentiated cells in the sites necessary in each case.

From U.S. Pat. No. 5,192,312 (Orton), a colonization process is already known in which an implantable human heart valve is treated with fibroblast growth factor and then colonized with an amount of fibroblasts which is supposed to make the implant nonimmunogenic. The preparation containing growth factors prevents this aim, since a primary masking with exogenous growth factor can lead to functional changes of the cells to be applied and an exogenously induced shift to a proliferatory phenotype. The exogenous addition of growth factors leads to internal competition mechanisms in signal communication, which finally lead to the fact that although many cells are formed, as a result of the growth factor these are not able to initiate necessary remodelling processes. The rapid completion of an autologized implant with respect to the supporting structures is thereby already prevented in vitro. An aftereffect in vivo is probable, since transformed cells can be formed. This has further important consequences after implantation in vivo, since allogenic and xenogenic matrices have immunogenic residual effects which lead to inflammatory reactions and the formation of calcification foci and thus to long-term transplant failure.

The invention is therefore based on the problem that a biological tissue selected for a transplantation and foreign to the transplantation recipient, in particular an allogenic or xenogenic material, is to be reacted to give a recipient-tolerable immunologically acceptable bioartificial transplant.

Furthermore, a process should be provided which produces mechanically more stable, naturally more similar transplants. The transformation process should proceed in a manner which is as controlled as possible with simultaneous stimulation and acceleration of the natural reconstruction.

For the solution of this problem, it is proposed according to the invention that in a process for the production of a bioartificial transplant from a biological tissue intended for transplantation and recipient-tolerable cells applied thereto,

recipient-tolerable cells which comprise at least selected cells capable of remodelling of the carrier structures are added in a conditioning medium to an autologous, allogenic or xenogenic tissue intended for transplantation, present in native form and not pretreated with exogenous growth factors,

the treatment of the transplant is continued until an extensive transformation of the original native tissue into a tissue essentially containing the recipient-specific cells added has been achieved.

“Cells capable of remodelling of the carrier structures” is understood as meaning those which contribute to secreting new tissue matrix and preferably also removing dead cells. This type includes, depending on the tissue type, various cells, e.g. fibroblasts and connective tissue cells, and their precursor cells from preferably autologous stem cells.

The cells capable of remodelling include in the cardiovascular field, for example, the smooth muscle cells. Generally, for example, macrophages are also included.

The cells mentioned promote and accelerate tissue transformation; as a rule they make possible the transformation thereby firstly, since otherwise other processes (calcification, rejection) would temporally “overtake” and in this manner prevent the tissue regeneration or tissue reconstruction.

The stimulus for tissue transformation can also be carried out by a specific inflammatory stimulus, which stimulates processes for tissue healing. The cells capable of remodelling can therefore also or in some cases be cells which can release inflammatory mediators. The tissue healing is then accompanied by an accelerated tissue reconstruction. The inflammatory mediators, however, can also be additionally added when using other cells capable of remodelling. This is then in particular carried out in a temporally restricted manner, such that a controllable healing-stimulating inflammatory process is initiated.

Among the stem cells are counted: bone marrow cells, (mesenchymal) cells originating from fatty tissue, tissue-specific stem cells, stem cells from peripheral blood, organ-specific stem cells, and cells after autologous nucleus transfer, for example endogenous muscle cell nuclei in fibroblasts (with trans-differentiation taking place).

The invention is based on the fundamentally novel concept of controlled tissue regeneration in vitro. Other than in the processes previously used, the native cells of the tissue intended for transplantation are neither removed as previously customary nor necessarily destroyed artificially. The tissue is rather subjected in a suitable device, which can be a customary colonization reactor, to artificial “wound healing”; in this process stimulation to newly growing cells is already primarily exerted by tissue-endogenous mediators.

A tissue in the native state is understood as meaning a tissue as dissected, i.e. removed from the xenogenic or allogenic donor. The cells present in the tissue, which find themselves in a state of dying from dissection or removal, are not removed, according to the principles of this invention, before the further treatment in separate process steps.

By means of the addition of cells which are tolerable for the recipient and matching the tissue type, the originally foreign transplant is gradually transformed during the treatment phase to give a bioartificial transplant which is completely immunotolerable for the recipient.

The invention is based on the realization that the removal or alternatively aggressive destruction of the original native cells of a foreign allogenic or xenogenic tissue has made recolonization difficult, namely in particular also because a stimulus emanating from these cells for the natural cell renewal which is continuously going on in every body is lost. In particular, important key factors for the finally necessary matrix reconstruction and for efficient de novo matrix synthesis were removed thereby.

The acellularization additionally caused a considerable destabilization, which, however, is urgently necessary with respect to a clinically necessary good initial stability for implantation purposes. The invention solves these problems.

As long as the foreign xenogenic or allogenic tissue intended for transplantation and colonized with native cells is left in its native state, on culture or incubation of the tissue in a conditioning environment consisting, for example, of nutrient medium cell mediators are released by cells of the transformed tissue which favor natural transformation (endogenous stimulus). The mediators divide in certain ways within the tissue and migrate into the conditioning medium to a small part. If now, during the culture of the tissue intended for transplantation, which is still provided with its native cells, new recipient-tolerable cells are added batchwise or continuously to the conditioning medium, these are included in the transformation process and with time replace the native cells which are gradually additionally drawn off during exchange of the conditioning medium. In this case, it is essential that the recipient-tolerable cells at least additionally include selected cells capable of remodelling of the carrier structures, e.g. connective tissue cells or fibroblasts. In addition, further recipient-tolerable cells can be present. The person skilled in the art can select the cells to be used in each case according to the information and explanations made above adapting the tissue type to be transformed.

Fundamentally, it is indeed known that natural—alternatively nonacellularized, for example non-denatured, allogenic transplants can be colonized on their surface by endothelial cells. This also takes place spontaneously in vivo after transplantation if the endothelial cells colonize an allogenic or xenogenic transplant in the body. Such an endothelialization, however, does not lead to actual transformation or to “remodelling” of the transplant tissue. Owing to immunological processes, starting calcification processes commence quite soon on the foreign (and foreign-remaining) tissue of individual focus points (calcification foci). The transplanted tissue or organ in this case becomes damaged to a greater and greater extent and finally functionally inactive in the course of time.

Animal experiments show that, for example, heart valves already spontaneously endothelialize within 24 to 48 hours. In this case, however, the tissue is not reconstructed but compressed. The endothelial cells remain physiologically on the surface. As L. Maxwell, J. G. Gavin, B. G. Barrett-Boyes have investigated in “Differences between heart valve allografts and xenografts in the incidence and initiation of dystrophic calcification”, Pathology (1989, 21, 5-10), the presence of residual donor cells leads to calcification nests, which finally bring about a valve failure.

The rejection and calcification of the transplanted tissue or organ can be avoided by the process according to the invention as, even before transplantation, remodelling in vitro is carried out, in which the tissue intended for transplantation is largely reconstructed.

For this, it is necessary that the recipient-specific cells used in the course of the process additionally comprise cells capable of remodelling. These include, inter alia, the fibroblasts, which can be induced by environmental stimuli to secrete new matrix and to promote the removal of old cells. Other cell types can additionally be used—mixed with the fibroblasts, in various layers or areas of the tissue.

Preferably, the recipient-tolerable cells are added once at the start of the culture, i.e. the treatment of the transplant, repeatedly at intervals or continuously within the medium.

In this case, the recipient-tolerable cells can be added dropwise or brushed onto the native tissue to be transformed, or added continuously or batchwise with the conditioning medium.

The recipient-tolerable or recipient-specific cells to be added to the transplant to be transformed can in certain embodiments be added mixed with a biologically tolerable adhesive, which in particular can contain fibrin, collagen adhesive proteins from mussels or synthetic adhesive proteins, or in a culture medium suspension.

The treatment of the transplant can be carried out with repeated exchange or under continuous flow of the medium, which can be a customary culture medium.

The transformation is preferably assisted mechanically in that the culture medium rinsing the tissue is stirred and a liquid flow is present for the transportation of new recipient-tolerable cells, which additionally washes away in the transformation of rejected/replaced cells. The tissue can be washed in between—once or at intervals, by means of which a mechanical stimulus is exerted which favors the detachment of cells to be replaced.

It is therefore essential for the invention that the treatment of the transplant with the recipient-tolerable cells in the conditioning medium is continued with repeated exchange or under continuous flow of the medium until a substantial reconstruction of the original native tissue into such a tissue has taken place which essentially only contains the recipient-specific cells used for the colonization.

The treatment of the native tissue in the conditioning medium with recipient-tolerable cells, the conditioning medium either being continuously recirculated or exchanged several times, corresponds to a colonization known per se of an underlying matrix with cells, such as is known in the prior art and can be carried out in various variants.

The conditioning medium used can be a customary cell culture nutrient medium which can optionally be provided with various additives. Nutrient media suitable for this are known to the person skilled in the art. Recipient-specific cells are introduced into the conditioning medium, either continuously or in a number of batches.

Recipient-specific cells are understood as meaning cells which are autologous or immunologically compatible or tolerable for the recipient. It is also possible to add various types of cells at different colonization or treatment times so that different cell layers of various cells can be built up on the tissue. Mixtures of different cells can furthermore be supplied to the tissue. Furthermore, various cells can be applied topically, for example different cells to the upper side and the underside of a skin transplant or different cells to the inside and the outside of a tubular vessel.

Possible recipient-tolerable cells are fundamentally all body cells, for example—depending on the underlying substrate—also those described below:

connective tissue cells (inter alia, fibroblasts, fibrocytes), muscle cells (myocytes), endothelial cells, skin cells (inter alia, keratinocytes), cells differentiated to give organ cells (heart cells, kidney cells, etc.), preferably in structured organs with a collagen structure, generally all cells which can usefully be supplied for the reconstruction of a specific tissue intended for implantation. Also suitable are the precursor cells, preferably from autologous stem cells of the recipient. The stem cells include those already mentioned above.

The tissue or the transplant to be transformed which is initially present in the native state, as removed, and is then transformed in the course of the process, can fundamentally be any transplantable tissue. In particular, these include: generally vessels, aortas, veins, aortal valves, heart valves, organ parts and whole organs, pieces of skin, tendons, cornea, cartilage, bone, larynx, heart, trachea, nerves, meniscus, intervertebral disk, ureters, urethra, bladder, inner ear ossicles, ear and nose cartilage, joint cartilage, connective tissue, fatty tissue, glandular tissue, nerves, muscles, inter alia.

For the reconstruction of the tissue with the aid of recipient-tolerable cells, cells or mixtures of cells are in each case selected which adapt to the respective tissue type. The recipient-tolerable, allogenic or xenogenic cells, which are preferably autologous or genetically modified and thereby rendered recipient-specific, comprise, in addition to the fibroblasts or connective tissue cells which are essential to the invention, those cells which are suitable for reconstruction of the desired tissue, and alternatively additionally those which can additionally stimulate and/or control the tissue transformation, such as, for example, cells producing cellular factors and/or cells having a chemotactic influence, among these especially cells from the family consisting of the leukocytes (lymphocytes, platelets, macrophages, mast cells, granulocytes, that is, for example, all forms of white blood corpuscles, granulocytes, lymphocytes, macrophages, monocytes, bone marrow cells, spleen cells, memory cells, thymus cells, and peripheral or central stem cells (from blood and bone marrow) or stem cells from fatty tissue, preferably pluripotent stem cells.

In the case of heart valves, fibroblasts or myofibroblasts, muscle cells and/or endothelial cells are preferably employed, in the case of skin transplants keratinocytes, cells of mesodermal origin (mesodermal cells) and optionally skin appendages.

An important aspect of the invention consists in the fact that the ideally autologous fibroblasts can mutate from a resting to an active phenotype through the signal action of the donor cells initially remaining, but dying in vitro. This has important consequences for the gene expression of the recipient-specific or recipient-tolerable cells, which in fact are also obtained from healthy tissue in a resting phenotype. In vitro, a “disease state” and therewith subsequently a “healing state” is then induced. In this context, the cooperation with ideally recipient-endogenous or recipient-specific helper cells can act to an increased and permissive extent. The recipient-tolerable cells which are employed for the tissue transformation therefore preferably also comprise macrophages, but also blood platelets, and immunocompetent cells such as lymphocytes.

It is central to the invention that the treatment is continued until a substantial, if not virtually complete, transformation is achieved or insofar as it was initiated, therewith a continuation of the continuous transformation in vitro is initiated.

A significant advantage of the invention results from the fact that implantations can take place more rapidly. In the conventional method, the foreign cells were firstly drawn off. In the course of this, the matrix was considerably weakened mechanically. Recolonization was then carried out, which demanded a period of at least 24 to 96 hours. The stability of the matrix gradually increased during the recolonization, but finally only up to about 70-80% of the starting value (e.g. measured by tensile stress). The process according to the invention makes possible a tissue transformation within about 4 days (3 to 6 days), the mechanical stability remaining approximately unchanged over the entire period. Since the tissue already initially corresponds to a physiological stability and load-bearing capacity, the danger of ruptures in the initial period after implantation is reduced considerably. The transformation is continued in the body in vivo (after implantation).

Before the treatment with the recipient-tolerable cells, the autologous, allogenic or xenogenic tissue intended for transplantation, which is present in native form, should be sterilized. In particular in the case of xenogenic tissues, this has to take place since it should be safely excluded that foreign viruses and bacteria are additionally introduced into the freshly produced bioartificial transplant. In the case of allogenic starting tissues too, disease transmission should be safely excluded. It should only be possible as an exception to transform autologous tissue for other use purposes. Here too, sterilization is useful, which, however, has to be less complicated.

A tissue in native form is understood as meaning such a tissue which has essentially been left as it has been removed. Native in this connection means natural, unaltered, nondenatured. On entry into the treatment phase with the recipient-tolerable cells, the tissue should still carry its native cells in order that the endogenous stimulus can be used for the transformation of the tissue. These cells, however, as already mentioned above, are in general already in the state of the start of dying because of the period of time elapsed for dissection and, if appropriate, transport.

The sterilization should be carried out as gently as possible. For the purposes of sterilization, rinsing can be carried out, for example, with a sterilizing solution or sterilization can be carried out using a gas (fumigation).

At present, sterilization by means of plasma ionization, in which a gas discharge takes place in the presence of H₂O₂, is regarded as particularly suitable. For this, an aqueous solution of hydrogen peroxide is injected into a sterilization chamber and vaporized. Under reduced ambient pressure, a low-temperature plasma is applied with the aid of radio frequency energy. By this means, an electrical field is generated which produces a plasma. In the plasma state, the hydrogen peroxide is cleaved with free-radical formation. The free radicals are the active species for the sterilization. This process leaves behind no toxic residues, since after conclusion of the reaction the free radicals react to give water, oxygen and other nontoxic products. The use of peroxides also corresponds to a natural process occurring in many cells (e.g. in macrophages).

If desired, the success of the sterilization can be specifically checked by testing, for example, for the presence of certain viruses or bacteria, which should be strictly prohibited, after the sterilization.

The tissue intended for transplantation can be exposed to additional non-denaturing process steps, e.g. rinsing, after its preparation before possibly necessary sterilization. Gentle freezing of the native transplant tissue is also possible provided relatively far-reaching tissue changes are avoided here.

In continuation of the invention, it is proposed that cellular mediators and/or factors or chemical mediators are additionally added to the conditioning medium, during the treatment with recipient-tolerable cells or thereafter. The action of certain factors has already being investigated, so that the person skilled in the art can specifically select and employ cell growth factors, cell-differentiating factors, chemotactic factors and others. In particular, the following can be used: neuropeptides: these can have the ability to activate mesenchymal cells. In the case of fibroblasts, proliferation and chemotaxis can be influenced. Among the suitable neuropeptides, the following may be mentioned in particular: neurokinin (neurokinin A (NKA)), substance P (SP), vasoactive intestinal peptide (VIP), calcitonin gene-related peptide (CGRP)); further mediators/factors which are mainly chemotactic and/or have a proliferation-controlling action which can be used are—depending on the cell and tissue type:

fibronectin (Fn), cytokines, such as interleukin-1-beta (IL-1 beta), interleukin-6 (IL-6), interleukin-8 (IL-8), interferons, such as interferon-gamma (IFN-gamma), granulocyte-macrophage colony-stimulating factor (GMCSF), transforming growth factor-beta 1 (TGF-beta 1)), osteogenic protein-1 (OP-1), recombinant human osteogenic protein-1 (rhOP-1), urokinase-type plasminogen activator (u-PA), PDGF (platelet-derived growth factor), in particular PDGF AA, PDGF AB, PDGF BB, HGF (hepatocyte growth factor), VEGF (vascular endothelial growth factor), FGF (fibroblast growth factor), ECGF (endothelial cell growth factor), glycoproteins, such as alpha-2-macroglobulin (alpha2M), Clara cell protein (CC-16), platelet factor 4, beta-thromboglobulin, neutrophil-activating peptide-1, furthermore also synthetic mediators, such as, for example, mannose 6-phosphate, adaptil and others.

The actual function of the individual mediators, factors, cofactors is known to the person skilled in the art from the area of isolated cells, so that he can select mediators/factors suitable for the respective purpose in the context of the invention described here.

In continuation of the invention, it is proposed that the process is carried out such that immunocompetent cells, in particular macrophages which release cellular mediators and/or factors into the conditioning medium, are added to the conditioning medium and/or tissue. In particular, the conditioning medium therefor can consist of autologous, i.e. recipient-endogenous, blood, herewith occasionally enriched or occasionally replaced by blood. By means of this, macrophages from the blood can adhere selectively to the tissue. The macrophages receive immunostimulatory stimuli from the tissue which is still not transformed or incompletely transformed and thereby release cell type-specific mediators which accelerate the reconstruction. Blood platelets lyse and release, for example, growth factors. The tissue reconstruction is stimulated, controlled and accelerated.

In a further development of the invention, it is proposed to carry out the process such that cellular mediators and/or factors are released into the conditioning medium or transferred to this from a culture of immunocompetent cells, in particular a macrophage culture. This culture can also contain lymphocytes or blood platelets. Furthermore, stem cells can also be added here.

The culture for the increased release of factors or mediators of suitable, for example immunocompetent, cells or the macrophage culture can be carried out in a bioreactor which is connected in a suitable manner to the reactor in which the bioartificial transplant is prepared and treated. Factors withdrawn from the bioreactor can be added in a suitable manner to the conditioning culture medium which is recirculating or added batchwise.

By means of a suitable bioreactor, a pressure- and stress-dependent remodelling can be carried out here, e.g. by pulsatile perfusion operation, for example in the case of vessels and heart valves, which has a very positive effect on the naturalness of the transformed tissue. It improves the expression corresponding to the normal physiology of the bioartificial tissue in vitro.

Alternatively, the macrophage culture, or the culture of other immunocompetent cells, can be kept separate from the conditioning medium during the steps consisting of the treatment with recipient-tolerable cells by means of a film, membrane or dividing wall which is permeable for the cellular mediators and/or factors, and the mediators and/or factors formed can be released continuously into the conditioning medium.

The treatment of the tissue intended for transplantation is in general carried out in a bioreactor in which the culture medium is held and optionally recirculated within a specific space. Within this space, a culture space for the culture of the immunocompetent cells or macrophages can be formed using a permeable dividing wall, such that the cell mediators and/or factors formed can migrate continuously into the conditioning medium. Alternatively, the immunocompetent cells can also be cultured separately and the cell culture products can be added to the bioreactor which is used for the tissue culture. In addition, the product (i.e. the organ or generally the tissue) can be perfused or coincubated for conditioning purposes with or without addition of recipient-specific whole blood or individual blood components (proteins, fibronectin, thrombin, fibrinogen, plasma, serum, cellular constituents). Immunocompetent cells which can be used are in particular the following:

all forms of white blood corpuscles, granulocytes, lymphocytes, macrophages, monocytes, bone marrow cells, spleen cells, memory cells, thymus cells.

Both abovementioned alternatives can also be combined by coculturing both immunocompetent, or immunomodulatory cells inside or outside the tissue bioreactor in order to produce specific mediators/factors, and at the same time also additionally adding naturally obtained or synthetic mediators/factors to the tissue culture medium.

The coculture of immunocompetent cells which produce mediators, factors, cofactors and release them into the conditioning medium is particularly advantageous, since mediators/factors particularly suitable for the respective purpose can be coproduced during a culture step which is anyway necessary, such that the use of additional expensive and less specific factors can be dispensed with.

The invention is described below with the aid of some examples:

### EXAMPLE 1

### Transdifferentiation of an Allogenic Cryoconserved Vein into an Autologous Artery

Cryoconserved allogenic veins are introduced into a bioreactor under sterile conditions without further treatment and perfused with ideally serum-free or autologous serum/plasma-enriched medium. Preexpanded autologous fibroblasts and smooth muscle cells originating from an artery are applied to the outside of the formerly cryoconserved vein. This takes place here by application in an (autologous) fibrin gel, collagen gel, in synthetic adhesive proteins from mussels by addition drop by drop or spreading of the cells mixed with the adhesive before the culturing or by addition drop by drop or spreading of a cell suspension in medium. Endothelial cells (optionally after a precolonization with myofibroblasts) are applied within the vascular lumen. This is carried out with slow rotation of the vessel within a bioreactor, where a bioreactor can be any device suitable for this. The fibroblasts are stimulated by the cell detritus of the dead cells to synthesize new matrix, to build up new tissue structures and to integrate to an increased extent into the tissue/the matrix. A multilayered muscle cell jacket is formed within a few days.

The arterialized (transformed) vessel is thus without loss of stability (such as customarily after the acellularization up to<20% initial strength) very rapidly ready for transplantation.

### EXAMPLE 2

### Transdifferentiation of a Xenogenic Cryoconserved Artery into an Autologous Human Artery

Xenogenic arteries are colonized without acellularization with autologous arterial vascular cells in analogy to the first example, but additionally without endothelial cells. The chimeric construct (transformed tissue) is rinsed with autologous blood. In this phase, macrophages adhere selectively to the exposed matrix. Lymphocytes receive immunostimulatory stimuli through the xenogenic matrix. Blood platelets lyse and release growth factors such as PDGF. After a time of action of about 4 hrs (sufficient for macrophage adhesion), the autologous blood is replaced again with plasma-enriched culture medium and recultured for several days (about 3-10). In this phase, an accelerated matrix turnover occurs due to the (autologous) myofibroblasts added for colonization. By means of pulsatile stresses, a directed pressure-controlled deposition of new matrix molecules and fibers takes place. The oriented integration of the newly formed cell associations is likewise made possible.

Alternatively, preparations of blood platelets (obtained at about 3000 g) and white blood corpuscles (1800 g) can be cocultured separately in different areas of the bioreactor or synchronously in a separate apparatus. In the latter case, the culture products of the tissue culture thus obtained are added to the actual tissue bioreactor.

1. A process for the production of a bioartificial transplant from a biological tissue intended for transplantation and recipient-tolerable cells applied thereto, characterized in that
recipient-tolerable cells which comprise at least selected cells capable of remodelling of the carrier structures are added in a conditioning medium to the autologous, allogenic or xenogenic tissue intended for the transplant, present in native form and not pretreated with exogenous growth factors,
the treatment of the transplant is continued until an extensive transformation of the original native tissue into a tissue essentially containing the added recipient-tolerable cells has been achieved.

2. The process as claimed in claim 1, characterized in that the cells capable of the remodelling of the carrier structures are connective tissue cells or their precursors, in particular fibroblasts or connective tissue precursor cells, preferably from autologous stem cells.

3. The process as claimed in claim 1, characterized in that the tissue to be transformed is a heart tissue and the cells capable of the remodelling are smooth muscle cells.

4. The process as claimed in claim 1, characterized in that the cells capable of the remodelling are macrophages.

5. The process as claimed in one of claims 1 to 4, characterized in that the recipient-tolerable cells are added once at the start of the culture, repeatedly at intervals or continuously within the medium.

6. The process as claimed in one of claims 1 to 5, characterized in that the recipient-tolerable cells are added dropwise or spread onto the native tissue to be transformed, or are added continuously or batchwise with the conditioning medium.

7. The process as claimed in one of claims 1 to 6, characterized in that the recipient-tolerable cells to be added are added mixed with a biologically tolerable adhesive, which in particular can contain fibrin, collagen or adhesive proteins, or in a culture medium suspension.

8. The process as claimed in one of claims 1 to 7, characterized in that the treatment of the transplant is carried out in the culture with repeated exchange or under continuous flow of the medium.

9. The process as claimed in one of claims 1 to 8, characterized in that cellular mediators and/or factors or chemical mediators are added to the conditioning medium during the treatment with recipient-tolerable cells.

10. The process as claimed in one of claims 1 to 9, characterized in that the process is carried out such that cells particularly capable and activatable for the release of cellular mediators and/or factors, in particular macrophages, are additionally added to the conditioning medium and/or to the tissue.

11. The process as claimed in claim 10, characterized in that the cells particularly capable and activatable for the release of cellular mediators and/or factors, in particular the macrophages, are held in a culture, preferably in a bioreactor, further preferably in the same bioreactor in which the transplant is also treated.

12. The process as claimed in claim 11, characterized in that the macrophage culture or corresponding cell culture is kept separate from the conditioning medium during the colonization or treatment with recipient-tolerable cells by means of a film, membrane or dividing wall which is permeable for the cellular mediators and/or factors, and the mediators and/or factors formed are released continuously into the conditioning medium.

13. The process as claimed in one of claims 1 to 12, characterized in that the autologous, allogenic or xenogenic tissue intended for transplantation and present in native form is first sterilized, preferably by rinsing with a sterile solution or by fumigation.

14. The process as claimed in claim 13, characterized in that the sterilization is carried out by means of plasma ionization with H₂O₂.

15. The process as claimed in one of claims 1 to 14, characterized in that the tissue intended for transplantation is exposed to additional non-denaturing process steps after its preparation, preferably before or after sterilization.

16. The process as claimed in one of claims 1 to 15, characterized in that the tissue intended for transplantation is additionally rinsed one or more times after its preparation, preferably before or after sterilization.

17. The process as claimed in one of claims 1 to 16, characterized in that the recipient-tolerable cells are autologous cells of the transplant recipient.

18. The process as claimed in one of claims 1 to 16, characterized in that the recipient-tolerable cells are allogenic or genetically modified allogenic cells selected as tolerable for the recipient.
